(19) United States
(12) Patent Application Publication
(10) Pub. No.: US 2026/0351379 A1
(43) Pub. Date: Nov. 5, 2026

(54) METHOD FOR CONSTRUCTING RADIOTHERAPY RESPONSE PREDICTION MODEL FOR GLIOMA, MEDIUM, AND PRODUCT

(71) Applicant: BEIJING INSTITUTE OF NEUROSURGERY, Beijing City (CN)

(72) Inventors: Yinyan WANG, Beijing City (CN); Zihan WANG, Beijing City (CN); Shuai LIU, Beijing City (CN); Jianhua HAN, Beijing City (CN); Hongbo BAO, Beijing City (CN); Zeya YAN, Beijing City (CN); Tao JIANG, Beijing City (CN)

(73) Assignee: BEIJING INSTITUTE OF NEUROSURGERY, Beijing City (CN)

(21) Appl. No.: 19/333,043

(22) Filed: Sep. 18, 2025

(30) Foreign Application Priority Data
Apr. 16, 2025 (CN) 202510472544.6

(51) Int. Cl. G16H 20/40; G16H 10/60; G16H 30/20; G16H 50/30; G16H 50/50

(52) U.S. Cl. CPC G16H 20/40; G16H 10/60; G16H 30/20; G16H 50/30; G16H 50/50

(57) ABSTRACT

Provided are a method for constructing a radiotherapy response prediction model for glioma, a medium, and a product, which relate to the technical field of medical devices. The present application achieves the prediction of the glioma radiotherapy response prediction by extracting and selecting features of magnetic resonance images (MRIs) based on radiomics, constructing a sample data set, and then training a linear regression model with the sample data set to obtain a trained linear regression model as the radiotherapy response prediction model for glioma.

### CROSS REFERENCE TO RELATED APPLICATION

This patent application claims the benefit and priority of Chinese Patent Application No. 2025104725446, filed with the China National Intellectual Property Administration on Apr. 16, 2025, the disclosure of which is incorporated by reference herein in its entirety as part of the present application.

### TECHNICAL FIELD

The present application relates to the technical field of medical devices, and in particular, to a method for constructing a radiotherapy response prediction model for glioma, a medium, and a product.

### BACKGROUND

Low-grade glioma (LGG) is a primary brain tumor originating from glial cells. Compared to high-grade glioma, LGG grows relatively slowly, and patients generally have a better overall prognosis after treatment. However, despite its lower malignancy, the biological heterogeneity of LGG makes treatment effects difficult to predict, posing significant challenges in clinical management. Radiotherapy, is an important adjuvant treatment following LGG surgeries, and its therapeutic effect varies considerably among patients. Increasing the radiotherapy dose may enhance treatment effects but also increases the risk of radiation-related toxicities, such as radiation necrosis. Conversely, reducing the radiotherapy dose may mitigate side effects but could lead to insufficient tumor control, affecting long-term therapeutic effects. This dilemma underscores the necessity of developing personalized therapeutic regimens based on individual characteristics of patients.

Current clinical assessments primarily rely on empirical judgments by physicians based on factors such as patient's age, gender, molecular pathological information of tumor, imaging manifestations, and clinical status. Particularly, the imaging manifestations and the judgment of the clinical status of the patient are often subjective, leading to inconsistent assessment criteria among different physicians and a lack of consistency and rigorous evidence to support radiotherapy response predictions. Existing empirical assessment methods depend on the molecular pathological information of tumor, which can be acquired only by obtaining a tumor tissue through craniotomy or biopsy and then performing sequencing analysis. As a result, non-invasive preoperative prediction remains unattainable.

At present, there are no effective tools to guide personalized radiotherapy strategies for LGG patients.

Therefore, establishing a model for predicting the glioma radiotherapy response pre-operatively and non-invasively is crucial for optimizing therapeutic strategies and enhancing postoperative radiotherapy effects.

### SUMMARY

An objective of the present application is to provide a method for constructing a radiotherapy response prediction model for glioma, a medium, and a product so as to enable accurate, preoperative, and non-invasive prediction of radiotherapy outcomes in glioma patients.

To achieve the above objective, the present application provides the following technical solutions.

In a first aspect, the present application provides a method for constructing a radiotherapy response prediction model for glioma, including the following steps:
acquiring a longitudinal multi-time-point magnetic resonance image (MRI) dataset, where the longitudinal multi-time-point MRI dataset includes multiple MRI sequences from glioma patients at three time points, namely, a preoperative time point, a postoperative but pre-radiotherapy time point, and a post-radiotherapy time point;
calculating, for each MRI sequence, a difference between products of transverse diameters and longitudinal diameters of maximum tumor cross sections in a postoperative but pre-radiotherapy image, and a post-radiotherapy image, and using the difference as a product difference corresponding to each MRI sequence;
determining a radiosensitivity corresponding to each MRI sequence based on the product difference corresponding to each MRI sequence;
extracting features from a preoperative image in each MRI sequence and performing feature selection to obtain a final feature set corresponding to each sequence;
constructing a sample dataset with the final feature sets corresponding to the MRI sequences as inputs and the radiosensitivities associated with respective sequence as labels; and
training a linear regression model with the sample data set to obtain a trained linear regression model as the radiotherapy response prediction model for glioma.

In a second aspect, the present application provides a computer-readable storage medium, storing thereon a computer program which, when executed by a processor, implements the method for constructing a radiotherapy response prediction model for glioma described above.

In a third aspect, the present application provides a computer program product, including a computer program which, when executed by a processor, implements the method for constructing a radiotherapy response prediction model for glioma described above.

According to specific embodiments provided in the present application, the present application has the following technical effects:

The present application provides a method for constructing a radiotherapy response prediction model for glioma, a medium, and a product. The present application achieves the prediction of the glioma radiotherapy response prediction by extracting and selecting features of MRIs based on radiomics, constructing a sample data set, and then training a linear regression model with the sample data set to obtain a trained linear regression model as the radiotherapy response prediction model for glioma.

### BRIEF DESCRIPTION OF THE DRAWINGS

To describe the technical solutions in the embodiments of the present application or in the related art more clearly, the following briefly describes the accompanying drawings required for describing the embodiments or the related art. Apparently, the accompanying drawings in the following description show some embodiments of the present application, and a person of ordinary skill in the art may still derive other accompanying drawings from these accompanying drawings without creative efforts.

FIG. 1 is a flowchart of a method for constructing a radiotherapy response prediction model for glioma provided by an embodiment of the present application; and

FIG. 2 is a diagram showing a predictive performance curve of a radiotherapy response prediction model for glioma provided by an embodiment of the present application.

### DETAILED DESCRIPTION OF THE EMBODIMENTS

The technical solutions in the embodiments of the present application will be described clearly and completely below with reference to the accompanying drawings in the embodiments of the present application. Apparently, the described embodiments are merely some rather than all of the embodiments of the present application. All other embodiments derived from the embodiments in the present application by those of ordinary skill in the art without creative efforts should fall within the protection scope of the present application.

To make the above objective, features, and advantages of the present application more obvious and easier to understand, the present application will be further described in detail with reference to the accompanying drawings and specific implementations.

Radiomics has emerged as a result of advancement in image pattern recognition technology, enabling the extraction of numerous objective quantitative features indiscernible to naked eyes from a medical image. These features include signal intensity distribution within tumor image, three-dimensional shape descriptors, texture characteristics, and features derived from wavelet transformations, etc. By providing a quantitative representation of the characteristics of solid tumors, radiomics offers a novel approach for the predictive analysis of clinical events related to the diagnosis and treatment of such disease. In glioma research, radiomics has demonstrated a broad application potential in multiple fields, particularly in the prediction of the therapeutic response. For example, Grossmann P & Kickingereder P et al. developed a radiomic model that can effectively identify recurrent glioblastoma patients likely to benefit from bevacizumab (anti-angiogenic therapy). Similarly, George E et al. constructed another radiomic model to identify recurrent glioblastoma patients likely to benefit from durvalumab (immunotherapy). These models provide critical support for personalized treatment and are expected to improve the treatment effects of patients.

MRI plays a crucial role in glioma diagnosis, treatment planning, and follow-up surveillance. Different MRI sequences, including T1-weighted (T1W), T2-weighted (T2W), contrast-enhanced T1-weighted (T1WC), and fluid attenuated inversion recovery (FLAIR), offer complementary information for the assessment of image features such as tumor structure, edema, and necrosis. In clinical practice, the therapeutic response of the glioma is typically assessed in accordance with Response Assessment in Neuro-Oncology (RANO) criteria which are employed to assess treatment effects by monitoring dynamic changes in tumor volume based on longitudinal multi-time-point MRI scanning. Given the importance of MRI in glioma management, radiomic models based on MRI data have emerged as promising tools for predicting the radiosensitivity of LGG and enable personalized therapeutic regimens.

According to the embodiments of the present application, a linear regression model is constructed on MRI data to predict the sensitivity of LGG to radiotherapy. The linear regression model may assist a physician in selecting the most suitable individualized therapeutic regimen, thereby improving the treatment effects and avoiding unwanted physical and psychological sufferings and financial burden.

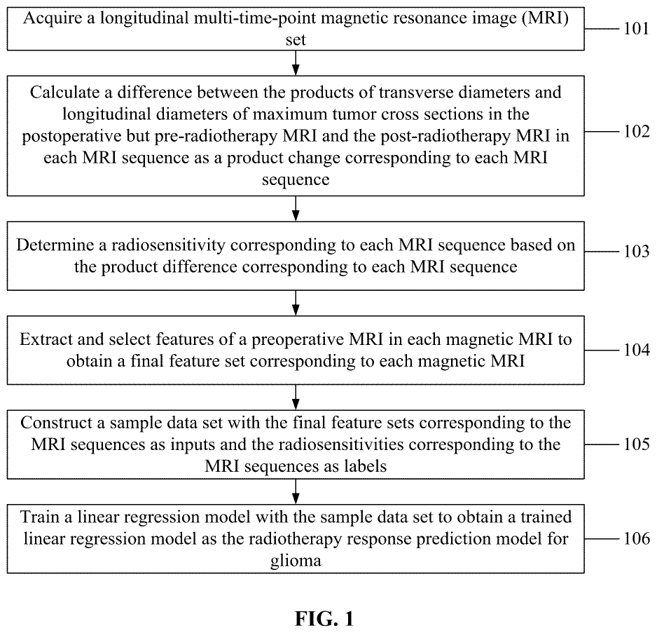

In an exemplary embodiment, as shown in FIG. 1, a method for constructing a radiotherapy response prediction model for glioma is provided, comprising steps 101 to 106.

In step 101, a longitudinal multi-time-point MRI dataset is acquired, comprising multiple MRI sequences (T1W, T2W, T1WC) from glioma patients at three time points: a preoperative time point, a postoperative but pre-radiotherapy time point, and a post-radiotherapy time point.

In step 102, a difference between the products of transverse diameters and longitudinal diameters of maximum tumor cross sections in the postoperative but pre-radiotherapy MRI and the post-radiotherapy MRI in each MRI sequence is calculated as a product difference corresponding to each MRI sequence.

In step 103, a radiosensitivity corresponding to each MRI sequence is determined based on the product difference corresponding to each MRI sequence.

In step 104, features of the preoperative MRI in each sequence are extracted and selected to form a final feature set corresponding to each MRI sequence.

In step 105, a sample data set is constructed with the final feature sets corresponding to the MRI sequences as inputs and the radiosensitivities associated with the respective sequence as labels.

In step 106, a linear regression model is trained with the sample data set to obtain a trained linear regression model as the radiotherapy response prediction model for glioma.

By implementing the above steps 101 to 106, a radiotherapy response prediction model for glioma can be established for predicting the glioma radiotherapy response.

In another exemplary embodiment of the present application, the above step 101 may be implemented in the following manner.

Longitudinal multi-time-point MRIs of LGG (Word Health Organization, WHO grades 1-2) are collected in a brain glioma database. The collected MRI sequences include T1W, T2W, and T1WC. These MRIs are collected at three specific time points: before surgery, within one week after surgery and before radiotherapy, and one month after surgery and the completion of radiotherapy.

The MRI technology can highlight different types of tissue or pathological features by using various scanning sequences and parameter settings. T1W, T2W, and T1WC are common the most commonly used imaging protocols, each playing a significant role in different clinical applications.

T1W images are acquired using relatively short repetition time (TR) and echo time (TE). T1 refers to the “spin-lattice relaxation time”, which is the time required for water protons to return to equilibrium through interactions with surrounding tissues. T1W imaging is typically used to display anatomical structures with high spatial resolution and contrast.

T2W images are acquired using relatively long TR and TE. T2 refers to the “spin-spin relaxation time”, which is the time it takes for water protons to lose phase coherence due to interactions with their surrounding environment, resulting in energy decay. T2W imaging is particularly suited for visualizing pathological changes such as cerebral edema, tumors, inflammation, and other abnormal tissue conditions.

The T1WC sequence is based on the standard T1W imaging, with the administration of a contrast agent (usually a gadolinium-containing contrast agent) to enhance signals intensity and improve the visibility of certain lesion. After intravenous injection, the contrast agent circulates through the bloodstream and accumulates in specific pathological areas, thereby increasing signal intensity in those regions. T1WC is widely used for tumor detection, localization, and evaluation of tumor vascularity, and is also helpful in identifying some other types of diseases, such as infection and spinal cord abnormalities.

In another exemplary embodiment of the present application, in step 102, based on the T2W images acquired before and after radiotherapy in step 101, the difference between the products of the transverse and longitudinal diameters of the maximum tumor cross sections is determined. This process specifically includes the following steps 201 to 203.

In step 201, the product of the transverse and the longitudinal diameter of the maximum tumor cross section in the postoperative but pre-radiotherapy MRI is calculated as a first product by the following formula:

P₁=L₁×W₁,
where P₁ represents the product of the transverse and the longitudinal diameter of the maximum tumor cross section in the postoperative but pre-radiotherapy MRI, namely the first product, L₁ represents the transverse diameter of the maximum tumor cross section in the postoperative but pre-radiotherapy MRI, and W₁ represents the longitudinal diameter of the maximum tumor cross section in the postoperative but pre-radiotherapy MRI, with W₁ being perpendicular to L₁.

In step 202, the product of the transverse and the longitudinal diameter of the maximum tumor cross section in the post-radiotherapy MRI is calculated as a second product using the following formula:

P₂=L₂×W₂,
where P₂ represents the product of the transverse and the longitudinal diameter of the maximum tumor cross section in the post-radiotherapy MRI, namely the second product, L₂ represents the transverse diameter of the maximum tumor cross section in the post-radiotherapy MRI, and W₂ represents the longitudinal diameter of the maximum tumor cross section in the post-radiotherapy MRI, with W₂ being perpendicular to L₂.

In step 203, calculate the product difference based on the first and the second product using the following formula:

$$Δ P = \frac{{P}_{1} - {P}_{2}}{{P}_{1}} \times 100 \% ,$$

where ΔP represents the product difference.

In another exemplary embodiment of the present application, the above step 103 is to compare the tumor volumes before and after radiotherapy according to the RANO criteria for determining the radiosensitivity. The specific assessment criteria are as follows.

If the tumor disappears completely after treatment, it is assessed as complete regression. If the reduction in tumor volume after treatment is ≥50%, it is assessed as partial regression. If the reduction in tumor volume is <50% and >25%, it is assessed as minor regression. If the tumor volume is between a reduction of 25% and an increase of 25%, it is assessed as stable disease. If the increase in tumor volume is ≥25%, it is assessed as progressive disease. Complete regression, partial regression, and minor regression are determined as sensitive to radiotherapy, and stable disease and progressive disease are determined as insensitive to radiotherapy.

In another exemplary embodiment of the present application, in step 104 described above, the preoperative MRI is segmented to obtain tumor habitat subregions. Radiomic features are then extracted from each of these subregions. The features from different subregions are fused to form a unified feature set. A final feature set corresponding to MRI sequence is obtained by using feature dimension reduction and selection methods. The following specific steps 301 to 306 are included.

In step 301, the preoperative MRI is segmented to obtain different tumor habitat subregions, including solid tumor subregion, cystic necrosis subregion, and edema subregion. The edema region refers to the area of swelling surrounding the tumor, defined within a preset range centered on the tumor.

In step 302, radiomic features are extracted from different tumor habitat subregions.

In step 303, radiomic features of different tumor habitat subregions are fused to form a unified feature set.

In step 304, the discriminative power of each radiomic feature in the unified feature set is calculated using a maximum relevance minimum redundancy (mRMR) algorithm, and the radiomic features are ordered by discriminability in a descending order to obtain a radiomic feature sequence.

In step 305, correlation analysis is performed on a first preset number of radiomic features in the radiomic feature sequence, and radiomic features meeting preset conditions are selected from the first preset number of radiomic features in the radiomic feature sequence to form a candidate radiomic feature set. The preset conditions are r>0.9 and p<0.05, with r being a correlation coefficient of Spearman's correlation test and p being a statistical test value.

In step 306, a second preset number of radiomic features are selected from the candidate radiomic feature set using the least absolute shrinkage and selection operator (LASSO) algorithm to form the final feature set. Exemplarily, the following 21 radiomic features are selected in this step.
1, original_ngtdm_Strength_CE-T1: original neighboring gray tone difference matrix strength_CE-T1;
2, wavelet-LLH_firstorder_RobustMeanAbsoluteDeviation_T2: wavelet-LLH first order robust mean absolute deviation_T2;
3, wavelet-LLH_glcm_ClusterProminence_T2: wavelet-LLH gray level concurrence matrix cluster prominence_T2;
4, original_ngtdm_Busyness_CE-T1: original neighboring gray tone difference matrix busyness_CE-T1;
5, wavelet-LLH_firstorder_Variance_CE: wavelet-LLH first order variance_CE;
6, wavelet-LLH_firstorder_RootMeanSquared_CE: wavelet-LLH first order root mean squared_CE;
7, wavelet-LLH_glcm_Autocorrelation_T2: wavelet-LLH gray level concurrence matrix autocorrelation_T2;
8, riginal_gldm_SmallDependenceLowGrayLevelEmphasis_CE-T1: original gray level dependence matrix small dependence low gray level emphasis_CE-T1;
9, log-sigma-5-0-mm-3D_gldm_DependenceEntropy_T1: log-sigma-5-0-mm-3D gray level dependence matrix dependence entropy_T1;
10, original_gldm_SmallDependenceHighGrayLevelEmphasis_CE-T1: original gray level dependence matrix small dependence high gray level emphasis_CE-T1;
11, log-sigma-5-0-mm-3D_glszm_ZoneVariance_T1: log-sigma-5-0-mm-3D gray level size zone matrix zone variance_T1;
12, wavelet-LLH_firstorder_RootMeanSquared_T2: wavelet-LLH first order root mean squared_T2;
13, log-sigma-5-0-mm-3D_glszm_ZoneVariance_CE: log-sigma-5-0-mm-3D gray level size zone matrix zone variance_CE;
14, log-sigma-5-0-mm-3D_glszm_ZonePercentage_T1: log-sigma-5-0-mm-3D gray level size zone matrix zone percentage_T1;
15, wavelet-LLH_glszm_GrayLevelNonUniformityNormalized_CE: wavelet-LLH gray level size zone matrix gray level nonuniformity normalized_CE;
16, log-sigma-5-0-mm-3D_glszm_ZonePercentage_CE: log-sigma-5-0-mm-3D gray level size zone matrix zone percentage_CE;
17, wavelet-LLH_girlm_ShortRunLowGrayLevelEmphasis_CE: wavelet-LLH gray level run length matrix short run low gray level emphasis_CE;
18, wavelet-LLH_glrlm_RunVariance_CE: wavelet-LLH gray level run length matrix run variance_CE;
19, log-sigma-5-0-mm-3D_ngtdm_Contrast_T2: log-sigma-5-0-mm-3D neighboring gray tone difference matrix contrast_T2;
20, wavelet-LLH_gldm_DependenceNonUniformity_T2: wavelet-LLH gray level dependence matrix dependence nonuniformity_T2; and
21, wavelet-LLH_glszm_LargeAreaEmphasis_T2: wavelet-LLH gray level size zone matrix zone variance large area emphasis_T2.

The suffixes T2, T1, and CE represent that the features are extracted from the T2W, the T1W, and the T1WC sequence, respectively. CE-T1 indicates that the feature is derived from the difference between the values extracted from the contrast-enhanced T1-weighted phase and the T1-weighted phase.

In another exemplary embodiment of the present application, the radiotherapy response prediction model for glioma obtained in the above step 106 is as follows:

R=0.520+0.055×M₁+0.009×M₂−0.043×M₃−0.028×M₄+0.016×M₅+0.091×M₆+0.022×M₇+0.018×M₈+0.042×M₉0.050×M₁₀−0.101×M₁₁+0.022×M₁₂+0.107×M₁₃−0.061×M₁₄−0.049×M₁₅−0.023×M₁₆+0.088×M₁₇−0.041×M₁₈+0.054×M₁₉−0.089×M₂₀+0.020×M₂₁,

where R represents a radiotherapy response sensitivity score; when R>0.5, a prediction result is determined as sensitive to radiotherapy or otherwise as insensitive to radiotherapy; and M₁ to M₂₁ represent original neighboring gray tone difference matrix strength_CE-T1, wavelet-LLH first order robust mean absolute deviation_T2, wavelet-LLH gray level concurrence matrix cluster prominence_T2, original neighboring gray tone difference matrix busyness_CE-T1, wavelet-LLH first order variance_CE, wavelet-LLH first order root mean squared_CE, wavelet-LLH gray level concurrence matrix autocorrelation_T2, original gray level dependence matrix small dependence low gray level emphasis_CE-T1, log-sigma-5-0-mm-3D gray level dependence matrix dependence entropy_T1, original gray level dependence matrix small dependence high gray level emphasis_CE-T1, log-sigma-5-0-mm-3D gray level size zone matrix zone variance_T1, wavelet-LLH first order root mean squared_T2, log-sigma-5-0-mm-3D gray level size zone matrix zone variance_CE, log-sigma-5-0-mm-3D gray level size zone matrix zone percentage_T1, wavelet-LLH gray level size zone matrix gray level nonuniformity normalized_CE, log-sigma-5-0-mm-3D gray level size zone matrix zone percentage_CE, wavelet-LLH gray level run length matrix short run low gray level emphasis_CE, wavelet-LLH gray level run length matrix run variance_CE, log-sigma-5-0-mm-3D neighboring gray tone difference matrix contrast_T2, wavelet-LLH gray level dependence matrix dependence nonuniformity_T2, wavelet-LLH gray level size zone matrix zone variance large area emphasis_T2, respectively.

After the radiotherapy response prediction model for glioma has been established, key radiomic features on the preoperative MRI of a glioma patient are extracted and input to the established radiotherapy response prediction model for glioma to obtain the predicted response of the patient to postoperative radiotherapy.

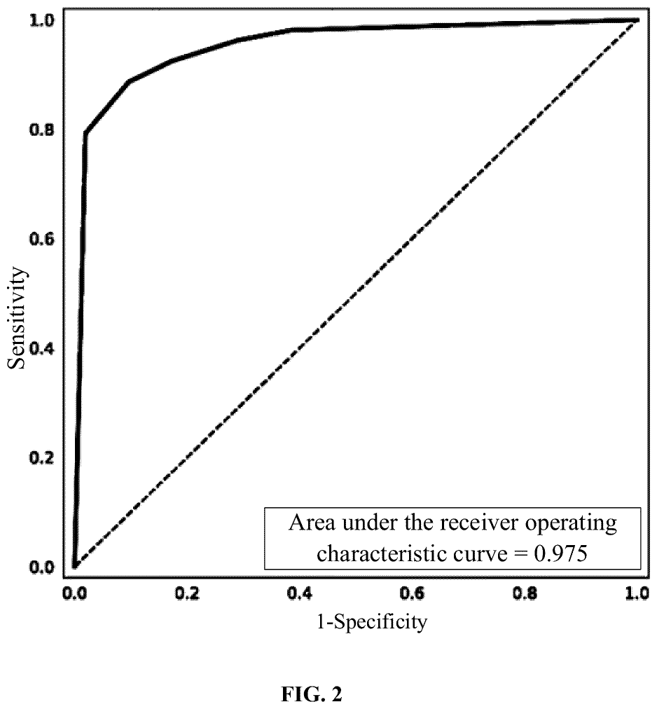

The performance of predicting the glioma radiotherapy response based on the above-mentioned radiotherapy response prediction model for glioma is as shown in FIG. 2. FIG. 2 shows a receiver operating characteristic curve of the radiotherapy response prediction model for glioma, where the X-axis is “1-specificity”, representing a proportion of negative samples that are wrongly identified as positive samples by the radiotherapy response prediction model for glioma, and the Y-axis is “sensitivity”, representing a proportion of positive samples correctly identified by the radiotherapy response prediction model for glioma. The area under the receiver operating characteristic curve is 0.975, indicating that the radiotherapy response prediction model for glioma exhibits excellent overall performance and is suitable for high-accuracy classification tasks.

Based on the above embodiments, this application achieves accurate assessment of a patient's response to radiotherapy by analyzing quantitative radiomic features extracted from MRI and applying a constructed predictive model. this application achieves accurate assessment of a patient's response to radiotherapy by analyzing quantitative radiomic features extracted from MRI and applying a constructed predictive model.

In an exemplary embodiment, a computer-readable storage medium is provided, storing a computer program which, when executed by a processor, implements the steps in the above method embodiments.

In another exemplary embodiment, a computer program product is provided, including a computer program which, when executed by a processor, implements the steps in the above method embodiments.

It is to be noted that user information (including, but not limited to, user device information, user personal information, etc.) and data (including, but not limited to, data for analysis, storage, and presentation, etc.) involved in the present application are information and data authorized by the user or fully authorized by each party.

Those of ordinary skill in the art may understand that all or some of the procedures in the methods of the above embodiments may be implemented by a computer program instructing related hardware. The computer program may be stored in a nonvolatile computer-readable storage medium. When the computer program is executed, the procedures in the embodiments of the above methods may be performed. Any reference to a memory, a database, or other media used in the embodiments of the present application may include a non-volatile and/or volatile memory. The nonvolatile memory may include a read-only memory (ROM), a magnetic tape, a floppy disk, a flash memory, an optical memory, a high-density embedded nonvolatile memory, a resistive random access memory (ReRAM), a magnetoresistive random access memory (MRAM), a ferroelectric random access memory (FRAM), a phase change memory (PCM), a graphene memory, etc. The volatile memory may include a random access memory (RAM) or an external cache memory. As an illustration rather than a limitation, the RAM may be in various forms, such as a static random access memory (SRAM) or a dynamic random access memory (DRAM).

The database in the embodiments of the present application may include at least one of a relational database and a non-relational database. The non-relational database may include a distributed database based on a blockchain, but is not limited thereto. The processor in the embodiments of the present application may be a general processor, a central processor, a graphics processor, a digital signal processor (DSP), a programmable logic device, and a data processing logic device based on quantum computing, but is not limited thereto.

The technical characteristics of the above embodiments can be employed in arbitrary combinations. To provide a concise description of these embodiments, all possible combinations of all the technical characteristics of the above embodiments may not be described; however, these combinations of the technical characteristics should be construed as falling within the scope defined by the specification as long as no contradiction occurs.

Several examples are used herein for illustration of the principles and implementations of the present application. The description of the foregoing embodiments is used to help understand the method of the present application and the core ideas thereof. Moreover, those of ordinary skill in the art can make various modifications in terms of specific implementations and scope of application in accordance with the teachings of the present application. In conclusion, the content of the present specification shall not be construed as a limitation to the present application.

What is claimed is:

1. A method for constructing a radiotherapy response prediction model for glioma, comprising:
acquiring a longitudinal multi-time-point magnetic resonance image (MRI) dataset, wherein the longitudinal multi-time-point MRI dataset comprises multiple MRI sequences from glioma patients at three time points, namely, a preoperative time point, a postoperative but pre-radiotherapy time point, and a post-radiotherapy time point;
calculating, for each MRI sequence, a difference between products of transverse diameters and longitudinal diameters of maximum tumor cross sections in a postoperative but pre-radiotherapy image and a post-radiotherapy image, and using the difference as a product difference corresponding to each MRI sequence;
determining a radiosensitivity corresponding to each MRI sequence based on the product difference corresponding to each MRI sequence;
extracting features from a preoperative image in each MRI sequence and performing feature selection to obtain a final feature set corresponding to each sequence;
constructing a sample data set with the final feature sets corresponding to the MRI sequences as inputs and the radiosensitivities associated with respective sequences as labels; and
training a linear regression model with the sample data set to obtain a trained linear regression model as the radiotherapy response prediction model for glioma.

2. The method for constructing a radiotherapy response prediction model for glioma according to claim 1, wherein calculating the difference between the products of the transverse diameters and the longitudinal diameters of the maximum tumor cross sections in the postoperative but pre-radiotherapy MRI and the post-radiotherapy MRI in each MRI sequence specifically comprises:
calculating the product of the transverse diameter and the longitudinal diameter of the maximum tumor cross section in the postoperative but pre-radiotherapy MRI as a first product by the following formula:
P₁=L₁×W₁,
wherein P₁ represents the product of the transverse diameter and the longitudinal diameter of the maximum tumor cross section in the postoperative but pre-radiotherapy MRI, namely the first product, L₁ represents the transverse diameter of the maximum tumor cross section in the postoperative but pre-radiotherapy magnetic resonance image, and W₁ represents the longitudinal diameter of the maximum tumor cross section in the postoperative but pre-radiotherapy MRI;
calculating the product of the transverse diameter and the longitudinal diameter of the maximum tumor cross section in the post-radiotherapy MRI as a second product by the following formula:
P₂=L₂×W₂,
wherein P₂ represents the product of the transverse diameter and the longitudinal diameter of the maximum tumor cross section in the post-radiotherapy MRI, namely the second product, L₂ represents the transverse diameter of the maximum tumor cross section in the post-radiotherapy MRI, and W₂ represents the longitudinal diameter of the maximum tumor cross section in the post-radiotherapy MRI; and
calculating the product difference based on the first product and the second product by the following formula:
ΔP=P₁−P₂/P₁×100%,
wherein ΔP represents the product difference.

3. The method for constructing a radiotherapy response prediction model for glioma according to claim 2, wherein determining the radiosensitivity corresponding to each MRI sequence based on the product difference corresponding to each MRI sequence specifically comprises:
if ΔP≥25%, determining the radiosensitivity as sensitive to radiotherapy, or otherwise, determining the radiosensitivity as insensitive to radiotherapy.

4. The method for constructing a radiotherapy response prediction model for glioma according to claim 2, wherein extracting and selecting the features from the preoperative MRI in each MRI sequence to obtain the final feature set corresponding to each MRI sequence specifically comprises:
segmenting the preoperative MRI to obtain different tumor habitat subregions, wherein the different tumor habitat subregions comprise a solid tumor subregion, a cystic necrosis subregion, and an edema subregion;
extracting radiomic features from the different tumor habitat subregions to obtain radiomic features of the tumor habitat subregions;
fusing the radiomic features of the tumor habitat subregions to form a unified feature set;
calculating a discriminability of each radiomic feature in the unified feature set using a maximum relevance minimum redundancy (mRMR) algorithm, and ordering the radiomic features by discriminability in a descending order to obtain a radiomic feature sequence;
performing correlation analysis on a first preset number of radiomic features in the radiomic feature sequence, and selecting radiomic features meeting preset conditions from the first preset number of radiomic features in the radiomic feature sequence to form a candidate radiomic feature set, wherein the preset conditions are r>0.9 and p<0.05, r being a correlation coefficient of Spearman's correlation test and p being a statistical test value; and
selecting a second preset number of radiomic features from the candidate radiomic feature set using a least absolute shrinkage and selection operator (LASSO) algorithm to form the final feature set.

5. The method for constructing a radiotherapy response prediction model for glioma according to claim 4, wherein the final feature set comprises: original neighboring gray tone difference matrix strength_CE-T1, wavelet-Low-Low-High (LLH) first order robust mean absolute deviation_T2, wavelet-LLH gray level concurrence matrix cluster prominence_T2, original neighboring gray tone difference matrix busyness_CE-T1, wavelet-LLH first order variance_CE, wavelet-LLH first order root mean squared_CE, wavelet-LLH gray level concurrence matrix autocorrelation_T2, original gray level dependence matrix small dependence low gray level emphasis_CE-T1, log-sigma-5-0-mm-3D gray level dependence matrix dependence entropy_T1, original gray level dependence matrix small dependence high gray level emphasis_CE-T1, log-sigma-5-0-mm-3D gray level size zone matrix zone variance_T1, wavelet-LLH first order root mean squared_T2, log-sigma-5-0-mm-3D gray level size zone matrix zone variance_CE, log-sigma-5-0-mm-3D gray level size zone matrix zone percentage_T1, wavelet-LLH gray level size zone matrix gray level nonuniformity normalized_CE, log-sigma-5-0-mm-3D gray level size zone matrix zone percentage_CE, wavelet-LLH gray level run length matrix short run low gray level emphasis_CE, wavelet-LLH gray level run length matrix run variance_CE, log-sigma-5-0-mm-3D neighboring gray tone difference matrix contrast_T2, wavelet-LLH gray level dependence matrix dependence nonuniformity_T2, wavelet-LLH gray level size zone matrix zone variance large area emphasis_T2.

6. The method for constructing a radiotherapy response prediction model for glioma according to claim 1, wherein the radiotherapy response prediction model for glioma is as follows:
R=0.520+0.055×M₁+0.009×M₂−0.043×M₃−0.028×M₄+0.016×M₃+0.091×M₆+0.022×M₇+0.018×M₈+0.042×M₉−0.050×M₁₀−0.101×M₁₁+0.022×M₁₂+0.107×M₁₃−0.061×M₁₄−0.049×M₃₅−0.023×M₁₆+0.088×M₁₇−0.041×M₁₈+0.054×M₁₉−0.089×M₂₀+0.020×M₂₁,
wherein R represents a radiotherapy response sensitivity score; when R>0.5, a prediction result is determined as sensitive to radiotherapy or otherwise as insensitive to radiotherapy; and M₁ to M₂₁ represent original neighboring gray tone difference matrix strength_CE-T1, wavelet-LLH first order robust mean absolute deviation_T2, wavelet-LLH gray level concurrence matrix cluster prominence_T2, original neighboring gray tone difference matrix busyness_CE-T1, wavelet-LLH first order variance_CE, wavelet-LLH first order root mean squared_CE, wavelet-LLH gray level concurrence matrix autocorrelation_T2, original gray level dependence matrix small dependence low gray level emphasis_CE-T1, log-sigma-5-0-mm-3D gray level dependence matrix dependence entropy_T1, original gray level dependence matrix small dependence high gray level emphasis_CE-T1, log-sigma-5-0-mm-3D gray level size zone matrix zone variance_T1, wavelet-LLH first order root mean squared_T2, log-sigma-5-0-mm-3D gray level size zone matrix zone variance_CE, log-sigma-5-0-mm-3D gray level size zone matrix zone percentage_T1, wavelet-LLH gray level size zone matrix gray level nonuniformity normalized_CE, log-sigma-5-0-mm-3D gray level size zone matrix zone percentage_CE, wavelet-LLH gray level run length matrix short run low gray level emphasis_CE, wavelet-LLH gray level run length matrix run variance_CE, log-sigma-5-0-mm-3D neighboring gray tone difference matrix contrast_T2, wavelet-LLH gray level dependence matrix dependence nonuniformity_T2, and wavelet-LLH gray level size zone matrix zone variance large area emphasis_T2, respectively.

7. A non-transitory computer-readable storage medium, storing thereon a computer program which, when executed by a processor, implements the method for constructing a radiotherapy response prediction model for glioma according to claim 1.

8. The non-transitory computer-readable storage medium according to claim 7, wherein calculating the difference between the products of the transverse diameters and the longitudinal diameters of the maximum tumor cross sections in the postoperative but pre-radiotherapy MRI and the post-radiotherapy MRI in each MRI sequence specifically comprises:
calculating the product of the transverse diameter and the longitudinal diameter of the maximum tumor cross section in the postoperative but pre-radiotherapy MRI as a first product by the following formula:
P₁=L₁×W₁,
wherein P₁ represents the product of the transverse diameter and the longitudinal diameter of the maximum tumor cross section in the postoperative but pre-radiotherapy MRI, namely the first product, L₁ represents the transverse diameter of the maximum tumor cross section in the postoperative but pre-radiotherapy magnetic resonance image, and W₁ represents the longitudinal diameter of the maximum tumor cross section in the postoperative but pre-radiotherapy MRI;
calculating the product of the transverse diameter and the longitudinal diameter of the maximum tumor cross section in the post-radiotherapy MRI as a second product by the following formula:
P₂=L₂×W₂,
wherein P₂ represents the product of the transverse diameter and the longitudinal diameter of the maximum tumor cross section in the post-radiotherapy MRI, namely the second product, L₂ represents the transverse diameter of the maximum tumor cross section in the post-radiotherapy MRI, and W₂ represents the longitudinal diameter of the maximum tumor cross section in the post-radiotherapy MRI; and
calculating the product difference based on the first product and the second product by the following formula:
ΔP=P₁−P₂×100%,
wherein ΔP represents the product difference.

9. The non-transitory computer-readable storage medium according to claim 8, wherein determining the radiosensitivity corresponding to each MRI sequence based on the product difference corresponding to each MRI sequence specifically comprises:
if ΔP≥25%, determining the radiosensitivity as sensitive to radiotherapy, or otherwise, determining the radiosensitivity as insensitive to radiotherapy.

10. The non-transitory computer-readable storage medium according to claim 8, wherein extracting and selecting the features from the preoperative MRI in each MRI sequence to obtain the final feature set corresponding to each MRI sequence specifically comprises:
segmenting the preoperative MRI to obtain different tumor habitat subregions, wherein the different tumor habitat subregions comprise a solid tumor subregion, a cystic necrosis subregion, and an edema subregion;
extracting radiomic features from the different tumor habitat subregions to obtain radiomic features of the tumor habitat subregions;
fusing the radiomic features of the tumor habitat subregions to form a unified feature set;
calculating a discriminability of each radiomic feature in the unified feature set using a maximum relevance minimum redundancy (mRMR) algorithm, and ordering the radiomic features by discriminability in a descending order to obtain a radiomic feature sequence;
performing correlation analysis on a first preset number of radiomic features in the radiomic feature sequence, and selecting radiomic features meeting preset conditions from the first preset number of radiomic features in the radiomic feature sequence to form a candidate radiomic feature set, wherein the preset conditions are r>0.9 and p<0.05, r being a correlation coefficient of Spearman's correlation test and p being a statistical test value; and
selecting a second preset number of radiomic features from the candidate radiomic feature set using a least absolute shrinkage and selection operator (LASSO) algorithm to form the final feature set.

11. The non-transitory computer-readable storage medium according to claim 10, wherein the final feature set comprises: original neighboring gray tone difference matrix strength_CE-T1, wavelet-Low-Low-High (LLH) first order robust mean absolute deviation_T2, wavelet-LLH gray level concurrence matrix cluster prominence_T2, original neighboring gray tone difference matrix busyness_CE-T1, wavelet-LLH first order variance_CE, wavelet-LLH first order root mean squared_CE, wavelet-LLH gray level concurrence matrix autocorrelation_T2, original gray level dependence matrix small dependence low gray level emphasis_CE-T1, log-sigma-5-0-mm-3D gray level dependence matrix dependence entropy_T1, original gray level dependence matrix small dependence high gray level emphasis_CE-T1, log-sigma-5-0-mm-3D gray level size zone matrix zone variance_T1, wavelet-LLH first order root mean squared_T2, log-sigma-5-0-mm-3D gray level size zone matrix zone variance_CE, log-sigma-5-0-mm-3D gray level size zone matrix zone percentage_T1, wavelet-LLH gray level size zone matrix gray level nonuniformity normalized_CE, log-sigma-5-0-mm-3D gray level size zone matrix zone percentage_CE, wavelet-LLH gray level run length matrix short run low gray level emphasis_CE, wavelet-LLH gray level run length matrix run variance_CE, log-sigma-5-0-mm-3D neighboring gray tone difference matrix contrast_T2, wavelet-LLH gray level dependence matrix dependence nonuniformity_T2, wavelet-LLH gray level size zone matrix zone variance large area emphasis_T2.

12. The non-transitory computer-readable storage medium according to claim 7, wherein the radiotherapy response prediction model for glioma is as follows:
R=0.520+0.055×M₁+0.009×M₂−0.043×M₃−0.028×M₄+0.016×M₃+0.091×M₆+0.022×M₇+0.018×M₈+0.042×M₉−0.050×M₁₀−0.101×M₁₁+0.022×M₁₂+0.107×M₁₃−0.061×M₁₄−0.049×M₃₅−0.023×M₁₆+0.088×M₁₇−0.041×M₁₈+0.054×M₁₉−0.089×M₂₀+0.020×M₂₁,
wherein R represents a radiotherapy response sensitivity score; when R>0.5, a prediction result is determined as sensitive to radiotherapy or otherwise as insensitive to radiotherapy; and M₁ to M₂₁ represent original neighboring gray tone difference matrix strength_CE-T1, wavelet-LLH first order robust mean absolute deviation_T2, wavelet-LLH gray level concurrence matrix cluster prominence_T2, original neighboring gray tone difference matrix busyness_CE-T1, wavelet-LLH first order variance_CE, wavelet-LLH first order root mean squared_CE, wavelet-LLH gray level concurrence matrix autocorrelation_T2, original gray level dependence matrix small dependence low gray level emphasis_CE-T1, log-sigma-5-0-mm-3D gray level dependence matrix dependence entropy_T1, original gray level dependence matrix small dependence high gray level emphasis_CE-T1, log-sigma-5-0-mm-3D gray level size zone matrix zone variance_T1, wavelet-LLH first order root mean squared_T2, log-sigma-5-0-mm-3D gray level size zone matrix zone variance_CE, log-sigma-5-0-mm-3D gray level size zone matrix zone percentage_T1, wavelet-LLH gray level size zone matrix gray level nonuniformity normalized_CE, log-sigma-5-0-mm-3D gray level size zone matrix zone percentage_CE, wavelet-LLH gray level run length matrix short run low gray level emphasis_CE, wavelet-LLH gray level run length matrix run variance_CE, log-sigma-5-0-mm-3D neighboring gray tone difference matrix contrast_T2, wavelet-LLH gray level dependence matrix dependence nonuniformity_T2, and wavelet-LLH gray level size zone matrix zone variance large area emphasis_T2, respectively.
